(19) United States
(12) Patent Application Publication
(10) Pub. No.: US 2007/0299536 A1
(43) Pub. Date: Dec. 27, 2007

(54) Collagen Mimics

(76) Inventors: Felicia A. Etzkorn, Blacksburg, VA (US); Xiaodong Wang, Maricapa, AZ (US); Matthew Shoulders, Madison, WI (US); Nan Dai, Blacksburg, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 10/599,926

(22) PCT Filed: Apr. 14, 2005
(86) PCT No.: PCT/US05/12409

§ 371 Date: Jun. 18, 2007

Related U.S. Application Data
(60) Provisional application No. 60/561,919, filed on Apr. 14, 2004.

(51) Int. Cl. A61F 2/32; C07K 14/00

(52) U.S. Cl. 623/23.57; 530/350; 623/23.11

(57) ABSTRACT

Novel peptidomimetics are provided, which mimic collagen. Molecular structures of interest include for imparting the collagen-mimicking property are each of: Gly-Ψ[(E)CH═C]-Xaa-Ψ[(E)CH═C]-Yaa; Gly-Xaa-Ψ[(E)CH═C]-Yaa; Gly-Xaa-Yaa-Ψ[(E)CH═CH]; Gly-Ψ[(E)CH═C]-Xaa-Ψ[(E)CH═C]-Yaa; Gly-Xaa-Ψ(E)CH═C]-Yaa-Ψ[(E)CH═CH]; Gly-Ψ[(E)CH═C]-Xaa-Yaa-Ψ[(E)CH═CH] and Gly-Ψ[(E)CH═C]-Xaa-Ψ[(E)CH═C]-Yaa-Ψ[(E)CH═CH]. Xaa and Yaa each means a natural amino acid, Hyp or Flp. Amide bonds may be altered to create collagen mimics. Preferably a tripeptide polymer comprising at least about 60 (Gly-Pro-Hyp) repeating units and having molecular weight of at least about 40,000 is synthesized as a long, collagen-like material. The new synthetic collagen-like materials may have better resistance to degradation, better mechanical strength and/or better ability to fold than natural collagen.

### FIELD OF THE INVENTION

This invention relates to the design and synthesis of collagen-like materials. More particularly the invention relates to materials that mimic the biological structure and behavior of collagen, yet are resistant to degradation

### BACKGROUND OF THE INVENTION

Collagen is generally regarded as one of the most useful biomaterials due to its excellent biocompatibility and safety. Major uses of collagen as a biomaterial include applications of collagen in drug delivery systems and in tissue-engineering systems. However, insufficient supply, poor mechanical strength, and ineffectiveness in the management of infected sites are problems for natural collagen-based systems.

Collagen is a natural material having as its basic repeating unit, Gly-Pro-Hyp. In collagen, proline (i.e., 2-pyrrolidinecarboxylic acid with formula C₄H₈NCOOH) and glycine (i.e., aminoacetic acid with formula NH₂CH₂COOH) are predominant components. Collagen is a highly abundant fibrous protein present throughout the human body, constituting approximately 25% of all protein in the body. Collagen is the scaffolding material found in skin, bones, tendons, cartilage, blood vessels and nearly all organs where it serves to form a matrix for holding and supporting cells. Collagen contains three polyproline type II helix chains each coiling in a left handed manner and coiling with each other to form a right-handed super helix. Kramer R Z, Bella J, Mayville P, Brodsky B, Berman H M: Sequence dependent conformational variations of collagen triple-helical structure. Nat. Struct. Biol. 1999, 6:454-457. The unique triple helical structure of collagen results from its primary structure, which can be represented as (Xaa-Yaa-Zaa)₃₀₀, where 10 percent of Xaa is proline, 10-12 percent of Yaa is 4(R)-hydroxyproline, and Zaa is typically Gly. Bansal M, Ramakrishnan C, Ramachandran G N: In Proc. Indian Acad. Sci.: 1975:152-164; Ramachandran G N, Ramakrishnan C: Biochemistry of Collagen. N.Y., London: Plenum Press; 1976. The presence of Gly at every third amino acid position is one of the most important structural elements of the collagen triple helix, as Gly is the only amino acid small enough to fit into the highly compacted super helix at that position. However, the high occurrence of hydroxyproline and proline in collagen and interchain hydrogen bonds between C═O and N—H groups contribute to stabilization of collagen's unique triple helical structure. Bansal et al, supra; Ramachandran et al., supra. A typical molecule of collagen consists of around 300 units of Xaa-Yaa-Gly. This highly repeated sequence of collagen makes possible the polymerization of tripeptide monomers to prepare collagen analogues.

The existence of stable Xaa-Pro and Xaa-Hyp cis and trans amide conformational isomers leads to a significant challenge for folding collagen peptides. Bruckner P, Eikenberry E F, Prockop D J: Formation of the triple helix of type I procollagen in cellulo. A kinetic model based on cis-trans isomerization of peptide bonds. Eur J Biochem 1981, 118:607-613; Sarkar S K, Young P E, Sullivan C E, Torchia D A: Detection of cis and trans X-Pro peptide bonds in proteins by 13C NMR: application to collagen. Proc Natl Acad Sci USA 1984, 81:4800-4803; Dolz R, Engel J, Kuhn K: Folding of collagen IV. Eur J Biochem 1988, 178:357-366; Buevich A V, Dai Q H, Liu X, Brodsky B, Baum J: Site-specific NMR monitoring of cis-trans isomerization in the folding of the proline-rich collagen triple helix. Biochemistry 2000, 39:4299-4308; Xu Y, Hyde T, Wang X, Bhate M, Brodsky B, Baum J: NMR and CD spectroscopy show that imino acid restriction of the unfolded state leads to efficient folding. Biochemistry 2003, 42:8696-8703.

In native collagens, globular C-terminal domains initiate triple helix formation (Doege K J, Fessler J H: J. Biol. Chem. 1986, 261:8924-8935), but proline isomerization is still the slow step in collagen folding. Eyles S J, Gierasch L M: Multiple roles of prolyl residues in structure and folding. J Mol Biol 2000, 301:737-747. In an average 300 unit repeat of Xaa-Yaa-Gly, with 10% of Xaa and Yaa each being Pro or Hyp, there are thus 60 amides that can exist in cis or trans. The number of possible conformational states of one strand is thus 2⁶⁰, and this does not include the necessity of triple helix formation. Folding of collagen occurs in a processive fashion. Once the triple helix is formed, the trans conformation is stable within the folded helix. Thus proline isomerization is rate limiting in collagen folding. Bruckner et al., supra; Sarkar et al., supra; Dolz et al., supra; Buevich et al, supra; Xu et al., supra.

Significant research has been performed regarding both the unique structural features of collagen and its potential biomedical applications. Lee C H, Singla A, Lee Y M: Int. J Pharmaceutics 2001, 221:1-22.Collagen is generally regarded as one of the most useful biomaterials due to its excellent biocompatibility and safety. Major uses of collagen as a biomaterial include applications of collagen in drug delivery systems and in tissue-engineering systems.

Several researchers have studied mimics of biological collagen, including polypeptides of the type (Pro-Pro-Gly)ₙ and (Pro-Flp-Gly)ₙ (where Flp represents 4(R)-fluoroproline), and all D-amino acid peptides. Sakikabara S, Inouye K, Shudo K, Kishida Y, Kobayashi Y, Prockop D J: Biochem Biophys Acta 1973, 303:198-202; Holmgren S K, Bretscher L E, Taylor K M, Raines R T: A hyperstable collagen mimic. Chem Biol 1999, 6:63-70; Li C, McCarthy J B, Furcht L T, Fields G B: An all-D amino acid peptide model of alpha1(IV)531-543 from type IV collagen binds the alpha3beta1 integrin and mediates tumor cell adhesion, spreading, and motility. Biochemistry 1997, 36:15404-15410. In these collagen mimics, amide bonds were unaltered.

Problems of insufficient supply, poor mechanical strength, and ineffectiveness in the management of infected sites of natural collagen-based systems, have been pointed out. Friess W: Eur. J. Pharm. Biopharm. 1998,45:113-136. A conventional approach of injecting materials prepared from sharks into humans has posed immunologic problems. Some synthetic collagen-like materials have been synthesized, mainly involving a low number of repeating units (such as 8 to 20 repeating units). The most commonly used technique has been to couple tripeptide units of Pro-Hyp(OtBu)-Gly to a solid resin.

However, further improvements and solutions continue to be desired for mimicking biological collagen, while improving upon certain properties of biological collagen.

### SUMMARY OF THE INVENTION

The present invention is aimed towards preparation of self-assembling, biologically stable mimics of collagen via amide bond polymerization of appropriate monomers.

In the invention, amide bonds are altered (such as, e.g., replacement of two amino acids with one molecule including an alkene double bond; replacement of one or more Hyp-Gly, Pro-Gly, Pro-Hyp, or Pro-Pro amide bond(s) with alkene isostere(s) in a collagen peptide; etc.) to prepare collagen mimics.

In a preferred embodiment, the invention provides a polymeric material which comprises at least one peptidomimetic selected from the following:
(Gly-Ψ[(E)CH═C]-Xaa-Yaa)ₙ (1A)
(Gly-Xaa-Ψ[(E)CH═C]-Yaa)ₙ (1B)
(Gly-Xaa-Yaa-Ψ[(E)CH═CH])ₙ (1C)
(Gly-Ψ[(E)CH═C]-Xaa-Ψ[(E)CH═C]-Yaa)ₙ (2A)
(Gly-Xaa-Ψ[(E)CH═C]-Yaa-Ψ[(E)CH═CH])ₙ (2B)
(Gly-Ψ[(E)CH═C]-Xaa-Yaa-Ψ[(E)CH═CH])ₙ (2C)
(Gly-Ψ[(E)CH═C]-Xaa-Ψ[(E)CH═C]-Yaa-Ψ[(E)CH═CH])ₙ (3)
wherein Xaa and Yaa may be the same or different and represent a natural amino acid, Hyp or Flp; n means an integer (preferably n is 10 or more), such as, e.g., a polymeric material comprising a block copolymer of a peptidomimetic with a natural peptide; a polymeric material comprising a monomer as follows: 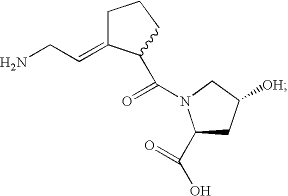
a polymeric material mimicking collagen (such as a polymeric material that is biocompatible and upon insertion into a region in a living patient where collagen at a previous time had been disposed, the inserted polymeric material provides at least one property of natural collagen); etc. Most preferably, the polymeric material is one in which the peptidomimetic comprises:
(Gly-Ψ[(E)CH═C]-Xaa-Yaa)ₙ (1A)
wherein Xaa is Pro and Yaa is Hyp. Another example of a polymeric material is one in which the peptidomimetic comprises:
(Gly-Ψ[(E)CH═C]-Xaa-Yaa)ₙ (1A)
wherein Xaa is Pro and Yaa is Pro. Another preferred example of an inventive polymeric material is one comprising a block polymer as follows: 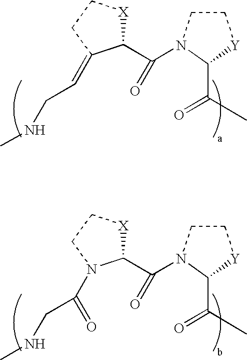
wherein a and b are integers between about 5 and 125, wherein a and b may be the same or different.

In another preferred embodiment, the invention provides a product comprising a polymeric material which is not naturally occurring, comprises alkene bonding and has a triple helix rope-like structure, such as, e.g., products wherein the polymeric material has one or more of: greater stability than natural collagen, and greater collagenase-resistance than natural collagen; greater ability to fold than natural collagen; products implanted or injected into a living organism; products having biology purity suitable for use in a living human patient; products not capable of producing a problematic immunologic reaction when injected into living human patients; etc. Examples of the polymeric material in such a product are, e.g., a polymeric material comprising at least one of the following: 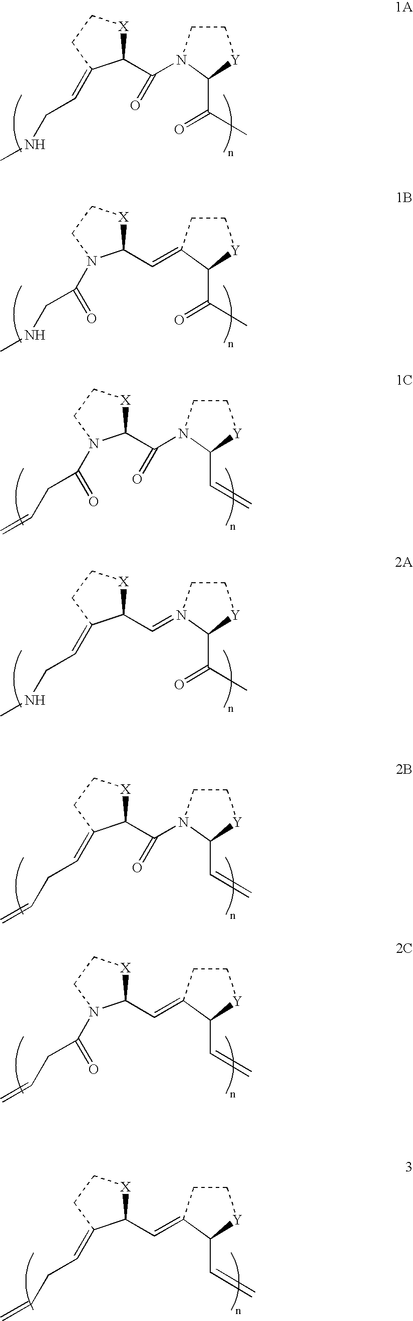
wherein n means an integer (preferably n is 10 or more); and other above-mentioned polymeric materials.

In another preferred embodiment, the invention provides a method of tissue replacement in a living organism, comprising: delivering into the living organism a product of the present invention or a polymeric material of the present invention.

A further embodiment of the invention provides a method of hip replacement, comprising: disposing in a living organism a product of the present invention or a polymeric material of the present invention.

In another embodiment, the present invention provides a biocompatible adhesive formed by a product of the present invention or a polymeric material of the present invention.

The invention also provides, in a further preferred embodiment, a method of biomineralization, comprising delivering, into a living organism, a product of the present invention or a polymeric material of the present invention.

In another preferred embodiment, the invention provides a method of drug delivery, comprising: disposing in a living organism a product of the present invention (or a polymeric material of the present invention) wherein a drug is included.

The invention in another preferred embodiment provides a method of synthesizing collagen-like peptides, comprising polymerization of a H-Gly-Ψ[(E)CH═C]-Pro-Hyp-OH monomer, such as, e.g., a synthesis method including polymerizing tripeptide units; a synthesis method wherein a (Gly-Pro-Hyp)ₜ polymer is synthesized wherein t is a number of repeating units of about 10 to 160; a synthesis method wherein a polymer comprising (Gly-Pro-Hyp) repeating units and having molecular weight of about 40,000 is synthesized; and other synthesis methods, etc.

### DETAILED DESCRIPTION OF A PREFERRED EMBODIMENT OF THE INVENTION

One or more properties mimicking that of biological collagen are exhibited by compounds selected from the group consisting of:
(Gly-Ψ[(E)CH═C]-Xaa-Yaa)ₙ (1A)
(Gly-Xaa-105 [(E)CH═C]-Yaa)ₙ (1B)
(Gly-Xaa-Yaa-Ψ[(E)CH═CH])ₙ (1C)
(Gly-Ψ[(E)CH═C]-Xaa-Ψ[(E)CH═C]-Yaa)ₙ (2A)
(Gly-Xaa-Ψ[(E)CH═C]-Yaa-Ψ[(E)CH═CH])ₙ (2B)
(Gly-Ψ[(E)CH═C]-Xaa-Yaa-Ψ[(E)CH═CH])ₙ (2C)
(Gly-Ψ[(E)CH═C]-Xaa-Ψ[(E)CH═C]-Yaa-Ψ[(E)CH═CH])ₙ (3)
wherein Xaa and Yaa may be the same or different and mean a natural amino acid, Hyp or Flp; Ψ means pseudo amide; (E) means entgegen as defined by IUPAC; n means an integer (preferably, an integer of 10 or more, especially an integer between about 10 and 250). A novel compound wherein Xaa is Pro and Yaa is Hyp has been synthesized (see Example 2 below). Compounds (1A), (1B), (1C), (2A), (2B), (2C), (3) also may be shown as follows: 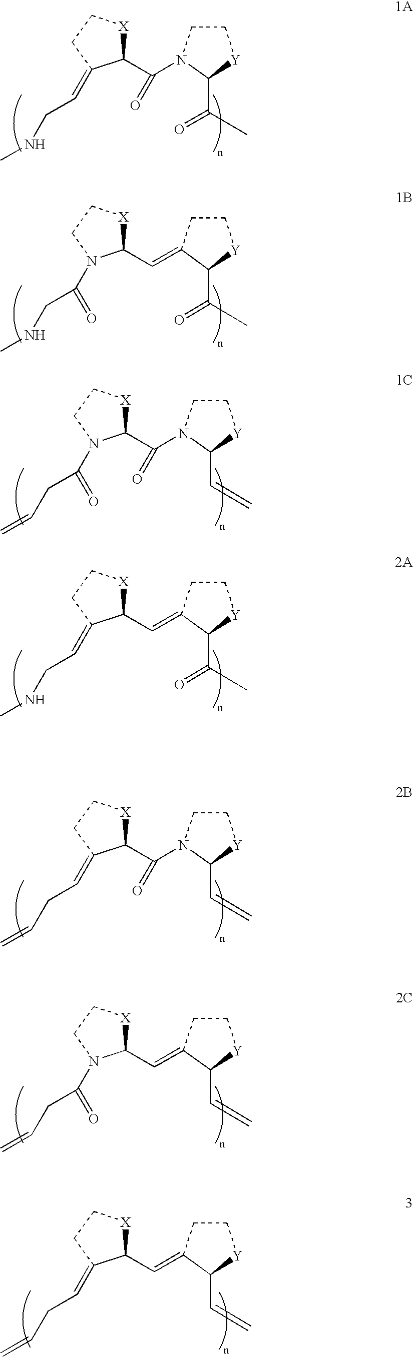
wherein n is as defined above, that is, n means an integer (preferably, an integer of 10 or more, especially an integer between about 10 and 250). Compounds according to above formulae (1A), (1B), (1C), (2A), (2B), (2C), (3) are referred to herein as “peptidomimetic” compounds or “peptidomimetics.”

“Natural amino acid” is used herein to refer to an amino acid that is one of the 20 natural amino acids. A natural amino acid may be in the Xaa and/or Yaa position(s) in inventive formulae (1A), (1B), (1C), (2A), (2B), (2C) and (3) herein.

“Hyp” has its usual meaning, 4(R)-hydroxyproline. Hyp may be in the Yaa position in inventive formulae (1A), (1B), (1C), (2A), (2B), (2C) and (3) herein.

“Flp” has its usual meaning, 4(R)-fluoroproline. Flp may be in the Yaa position in inventive formulae (1A), (1B), (1C), (2A), (2B), (2C) and (3) herein.

Compounds possessing properties mimicking biological collagen may be used in biomaterials applications, such as tissue replacement; injection into the human body (such as into the shoulder, hip, etc.); etc.; in drug delivery; as an adhesive that is biocompatible (such as, e.g., for use in hip replacement); in biomineralization; etc. The peptidomimetic compounds of the present invention (e.g., compounds according to formulae (1A), (1B), (1C), (2A), (2B), (2C), (3)), and enantiomers ((R-1A), (R-1B), (R-1C), (R-2A), (R-2B), (R-2C), (3)) where all amino acids and their replacements have the unnatural D-amino acid, R- or S-stereochemistry at the α-position, and the correspondingly opposite stereochemistry in any side chains, and the racemic material, i.e. a 1:1 mixture of natural and unnatural stereochemistry may be used in biomaterials applications, preferably, as a substitute for naturally-occurring collagen and in all applications which have been recognized for synthetic collagen. Compounds of inventive formula (1A) are preferred for use in the present invention, with compounds of inventive formula (1A) wherein Xaa is Pro and Yaa is Hyp or Pro being most preferred. Enantiomers (R-1A), (R-1B), (R-1C), (R-2A), (R-2B), (R-2C), (3) are as follows: 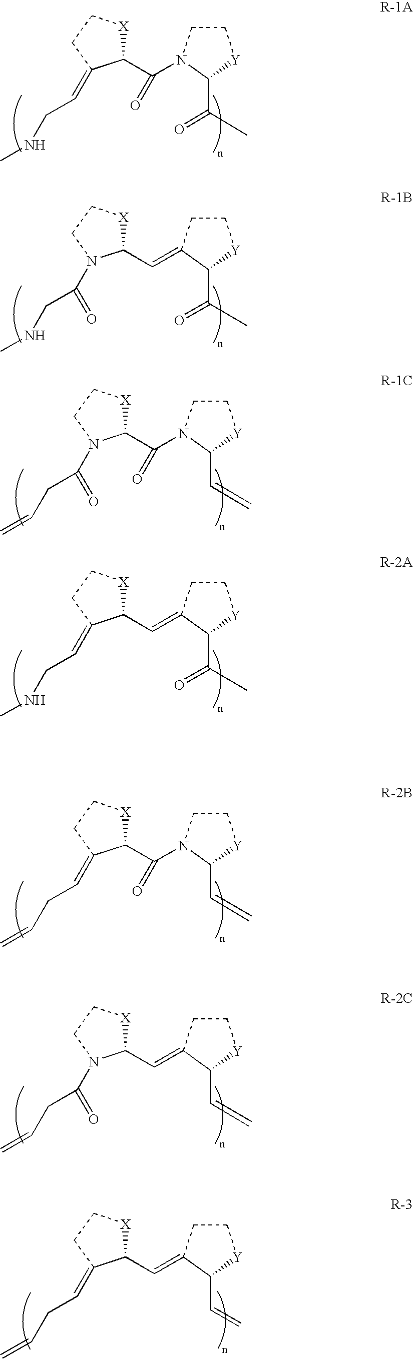
wherein in each of formula R-1A, R-1B, R-1C, R-2A, R-2B, R-2C and R-3, “n” means an integer (preferably, an integer of 10 or more, especially an integer between about 10 and 250).

The inventive peptidomimetics mimic the three helices in the tertiary structure of natural collagen. The peptidomimetics of the present invention may be more stable; fold better; and/or be more resistant to collagenase than naturally occurring collagen. The present invention advantageously provides alkene amide bond surrogates. The inventive alkene amide bond surrogates may provide one or more of the following: conformational control; resistance to peptidases; inhibition of collagenase (matrix metalloproteases); prevention of mucositis (such as in cancer therapy); and/or acting as a clinical marker of rheumatoid arthritis.

Examples of inventive compounds (1A), (1B), (1C), (2A), (2B), (2C) and (3) include, e.g., block copolymers of alkene isostere monomers with tripeptide monomers, compounds of the following formula II, and enantiomers where all amino acids and their replacements have the unnatural D-amino acid, R- or S-stereochemistry at the a-position. Such all D-amino acid analogues may have particular stability towards biological degradation with the enantiomeric right-handed triple helix supercoil producing similar macroscopic materials properties, yet interesting alternative biological properties. (Li C, McCarthy J B, Furcht L T, Fields G B: An all-D amino acid peptide model of alpha1(IV)531-543 from type IV collagen binds the alpha3beta1 integrin and mediates tumor cell adhesion, spreading, and motility. Biochemistry 1997, 36:15404-15410.)

In the invention, a compound according to inventive formula (1A), (1B), (1C), (2A), (2B), (2C) or (3) to peptidomimetics may be formed into a block copolymer including natural peptides, such as a block copolymer comprising a peptidomimetic of formula (1A), such as, e.g., the following example of a block copolymer of formula (II) wherein a peptidomimetic of formula (1A) is included: 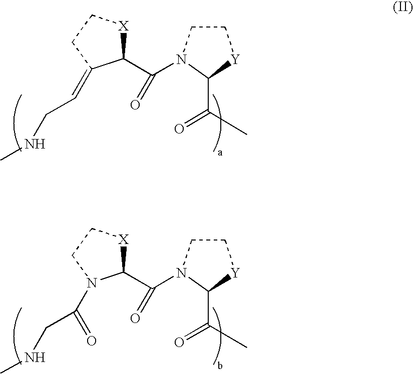
wherein a and b are integers, preferably between about 5 and 125, wherein a and b may be the same or different integer. Enantiomeric collagen mimic materials according to formula (II) are novel.

Also, inventive materials are provided in which are included block copolymers of mixtures of alkene isostere monomers with tripeptide monomers, of which the following formula is an example: 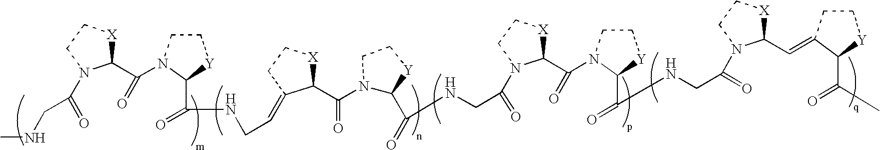
(Gly-Xaa-Yaa)ₘ(Gly-Ψ[(E)CH═C]-Xaa-Yaa)ₙ(Gly-Xaa-Yaa)ₚ(Gly-Ψ[(E)CH═C]-Xaa-Yaa)_{q}
wherein the above formula depicts a block copolymer of alkene isostere with natural peptides; m, n, p, and q are integers which may be the same or different.

Inventive materials also are provided for the enantiomeric case where all amino acids and their replacements have the unnatural D-amino acid (R- or S-stereochemistry) at the α-position according to the following formula: 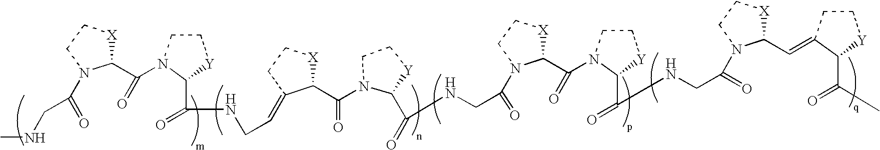
enantio-(Gly-Xaa-Yaa)ₘ(Gly-Ψ[(E)CH═C]-Xaa-Yaa)ₙ(Gly-Xaa-Yaa)ₚ(Gly-Xaa-Ψ[(E)CH═C]-Yaa)_{q}
wherein m, n, p, and q are integers which may be the same or different). Such all D-amino acid analogues may have particular stability towards environmental degradation with the enantiomeric left-handed triple helix supercoil producing similar macroscopic materials properties.

Generally, a preferred size for the inventive materials is a molecular weight of 40,000 or above, corresponding to (Gly-Pro-Hyp)ₙ polymers with about 160 repeating units. Inventive long collagen-like polymers may be assembled by polymerizing tripeptide units in solution.

Collagen-like peptides may be synthesized via polymerization of monomers such as Gly-Ψ[(E)CH═C]-Pro-Hyp. Because all the Gly-Pro amide bonds in collagen exist in the trans conformation, a route that affords the E monomer stereo selectively is desired. The present inventors recently had success in Ser-trans-Pro (E)-alkene isostere synthesis (Wang X J, Hart S A, Xu B, Mason M D, Goodell J R, Etzkorn F A: Serine-cis-proline and Serine-trans-proline Isosteres: Stereoselective Synthesis of (Z)- and (E)-Alkene Mimics by Still-Wittig and Ireland-Claisen Rearrangements. J. Org. Chem. 2003, 68:2343-2349) and herein are providing such a synthesis route to the Gly-Ψ[(E)CH═C]-Pro-Hyp monomer. Alkene amide bond surrogates provide not only conformational control but also resistance to peptidases.

The alkene isostere material is also likely to inhibit collagenase (matrix metalloproteases), and may represent a method for preventing mucositis in cancer therapy (Morvan F O, Baroukh B, Ledoux D, Caruelle J P, Barritault D, Godeau G, Saffar J L: An engineered biopolymer prevents mucositis induced by 5-fluorouracil in hamsters. Am J Pathol 2004, 164:739-746), or improving clinical markers of rheumatoid arthritis (Klimiuk P A, Sierakowski S, Latosiewicz R, Cylwik B, Skowronski J, Chwiecko J: Serum matrix metalloproteinases and tissue inhibitors of metalloproteinases in different histological variants of rheumatoid synovitis. Rheumatology (Oxford) 2002, 41:78-87).

The inventive synthetic collagen mimics may be used for studying the stability of collagen-like triple helical structures; for providing useful structural biomaterials; etc.

Some inventive Examples are set forth below, without the invention being limited to those Examples.

### EXAMPLE 1

### (Synthesis of the [Gly-Pro-Ψ[(E)CH═C]Hyp]ₙ Monomer)

The synthetic scheme for preparation of the Gly-Ψ[(E)CH═C]Pro amide bond isostere is shown in Scheme 1 below (which is analogous in certain principles to our previously described synthetic scheme in Wang et al., supra): 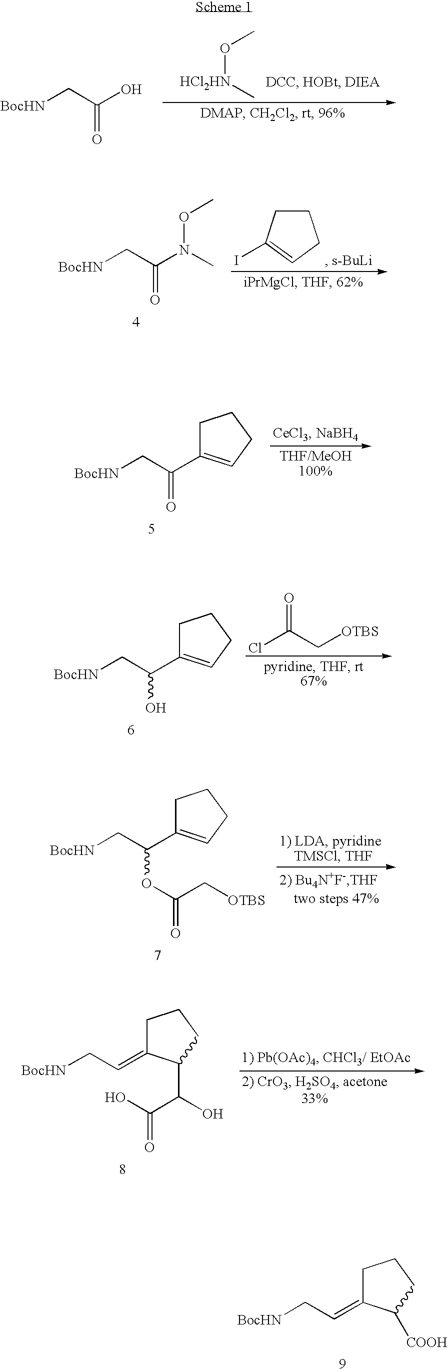
The general synthetic scheme in Scheme I above is applicable to all possible amino acids used in collagen mimetics. In order to obtain a pure enantiomer of 9, a chiral hydrogenation catalyst instead of CeCl₃ and NaBH₄ is used in the reduction of α,β-unsaturated ketone 5. Binaphthyl rhodium hydrogenation catalyst is mentioned, but other catalysts are possible. After preparation of 9, the monomer used for the amide bond polymerization may be prepared as displayed in Scheme 2.

In order to ascertain the best conditions for polymerization, the tripeptide H-Gly-Pro-Pro-OH was synthesized by standard solution-phase peptide synthesis. The tripeptide, H-Gly-Pro-Hyp-OH, with and without Hyp side chain protection were prepared and polymerized in solution using HBTU, HOBt, and DIEA in NMP at 55° C. for 7 days. Products were isolated by precipitation and characterized by ¹H NMR and GPC. Polymerization of the tripeptide isostere 12, with tbutyldimethylsilyl protection on the Hyp side chain, was unsuccessful under the same conditions. The protected monomer was polymerized with HATU, HOAt, and DIEA in NMP at 50° C. for 12 hours. Initial characterization by TLC and ¹H DMF indicate formation of a polymer 1a. Deprotection of the tert-butyl dimethyl silyl group may have occurred during polymerization, but nevertheless is expected to occur readily with standard fluoride conditions, either nBu₄NF or HF in CH₃CN to make collagen mimic 1a.

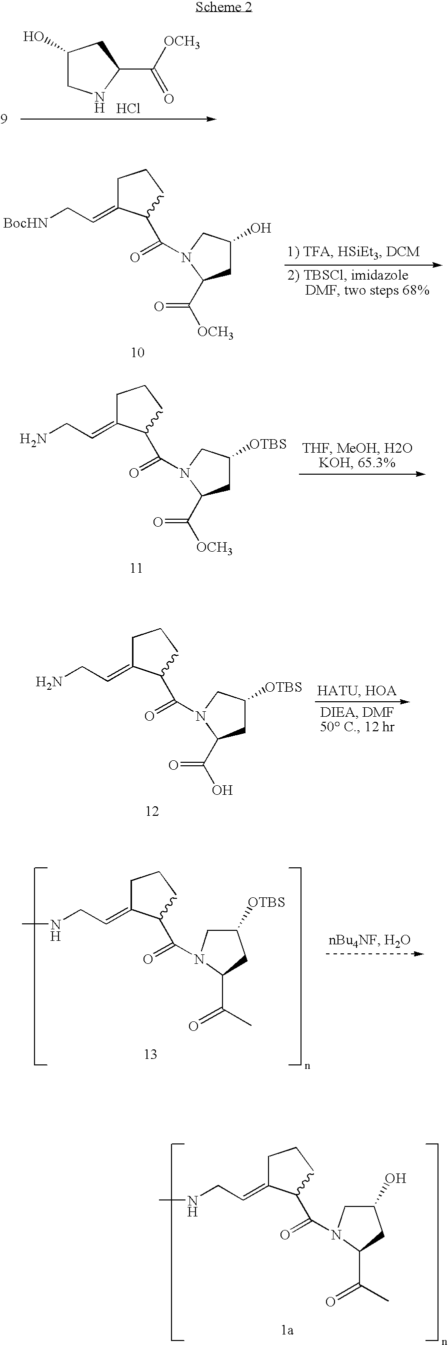

The synthesis of the monomer to make one example of material 2B is shown in Scheme 3.

Polymerization can be performed by the method shown in Scheme 2.

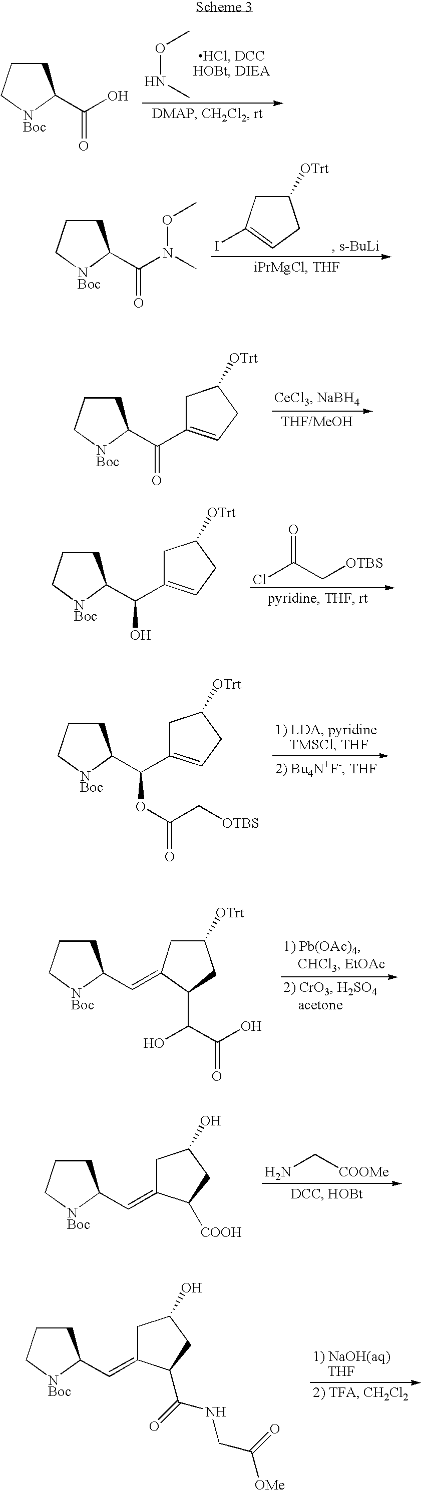

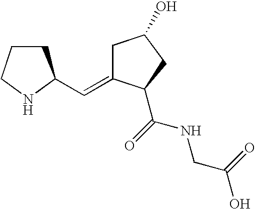

Synthesis of materials of type C, including 3, will be polymerized by ADMET (acyclic diene metathesis) catalysis. (Hopkins T E, Pawlow J H, Koren D L, Deters K S, Solivan S M, Davis J A, Gomez F J, Wagener K B: Chiral Polyolefins Bearing Amino Acids. Macromolecules 2001, 34:7920-7922.) An example is shown below in Scheme 4 for a peptidomimetic compound according to inventive formula (1C).

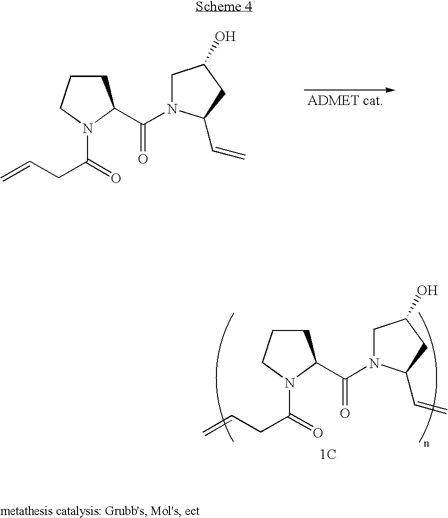

### EXAMPLE 2

### Synthesis of Monomer

A novel monomer H-Gly-Ψ[(E)CH═C]-Pro-Hyp-OH according to formula (IV) below was synthesized. 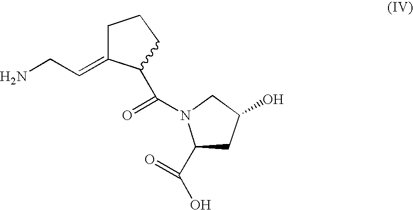
The novel monomer of above formula (IV) can be polymerized to make a collagen mimic. The monomer of formula (V), was synthesized by a novel method (see synthesis Example 1 above). The key to production of the trans isostere was the Ireland-Claisen rearrangement to produce 8 in above Scheme 1. The chirality of the alcohol 6 (Scheme 1) is transmitted to the cyclopentane ring during the Ireland-Claisen rearrangement. The extra carbon was then removed by oxidative decarboxylation to produce 9 (Scheme 1).

In summary, a racemic Gly-traits-Pro isostere according to the present invention was synthesized. Other isosteres according to the present invention may be similarly synthesized, by using appropriate starting materials.

### EXAMPLE 3

Experimentation was performed as follows.

General

Unless otherwise indicated, all reactions were carried out under N₂ in flame-dried glassware. THF and CH₂Cl₂ were dried by passage through aluminum. Anhydrous (99.8%) peptide synthesis grade DMF, NMP and diisopropylethylamine (DIEA) were purchased from Fluka Chemical Co. for solid phase synthesis. Brine (NaCl), NaHCO₃, and NH₄Cl refer to saturated aqueous solutions unless otherwise noted. Flash chromatography was performed on 32-63 μm or 230-400 mesh, ASTM silica gel with reagent grade solvents. NMR spectra were obtained at ambient temperature in CDCl₃ unless otherwise noted. Proton and carbon-13 NMR spectra were obtained at 500 and 125 MHz, respectively. Coupling constants J are given in Hertz.

Boc-Gly Weinreb amide (4) (Niel G, Roux F, Maisonmasse Y, Maugras I, Poncet J, Jouin P: Substrate-controlled Croylboration from N-(tert-Butoxycarbonyl)amino Aldehydes. J. Chem. Soc. Perkin Trans. 1 1994, 10:1275-1280.) N-Boc-Gly-OH (10.5 g, 60.0 mmol), N,O-dimethylhydroxylamine hydrochloride (11.1 g, 120 mmol) and DIEA (31.2 g, 240 mmol) were dissolved in 1:1 CH₂Cl₂/DMF (500 mL) and cooled to 0° C. 1-Hydroxy-1H-benzotriazole (HOBt, 11.0 g, 72.0 mmol), DCC (14.9 g, 72.0 mmol) and DMAP (ca. 100 mg) were added and the reaction was stirred for 24 h. The reaction was filtered to remove dicyclohexylurea and concentrated. The resulting slurry was diluted with 500 mL ethyl acetate and washed with NH₄Cl (2×100 mL), NaHCO₃ (2×100 mL) and brine (100 mL). The organic layer was dried on MgSO₄ and concentrated. Chromatography on silica with 20% EtOAc in hexane gave 12.6 g (96%) of 4 as a colorless plate-like crystal. m.p. 101-102° C. ¹H NMR δ 5.25 (br, s, 1H), 4.07 (d, J=3.7, 2H), 3.70 (s, 3H), 3.19 (s, 3H), 1.44 (s, 9H).

Ketone (5). To a solution of 1-iodocyclopentene[16] (2.91 g, 15.0 mmol) in 80 mL THF at −40° C. was added s-BuLi (1.3 M in cyclohexane, 23 ml, 30 mmol). The reaction was stirred at −40° C. for 3 h to generate cyclopentenyl lithium. In another flask, Boc-glycine Weinreb amide 4 (2.18 g, 10.0 mmol) was dissolved in 20 mL of dry THF, degassed and inerted under N₂. The solution was cooled to −15 to −10° C. and to the resulting slurry was charged with 4.9 mL of 2.0 M i-PrMgCV/THF (9.8 mmol) dropwise at −15 to −5° C. to afford a clear solution. After cooling to −78° C., the cyclopentenyl lithium solution was added via cannula to the deprotonated Weinreb amide solution. The mixture was stirred for 1 h at −78° C., quenched with NH₄Cl (10 mL), diluted with EtOAc (100 mL), washed with NH₄Cl (2×20 mL), NaHCO₃ (20 ml), brine (20 mL), dried over MgSO₄ and concentrated. Chromatography on silica with 10% EtOAc in hexane gave 1.40 g (62%) of ketone 5 as a yellowish solid. ¹H NMR δ6.81 (s, 1H), 5.36 (br, s, 1H), 4.29 (d, J=4.6, 2H), 2.56 (t, J=7.7, 4H), 1.92 (m, 2H), 1.43 (s, 9H). ¹³C NMR δ 192.9, 155.8, 144.6, 143.1, 79.7, 47.5, 34.2, 30.6, 28.4, 22.5. Anal. Calcd. for: C₁₂H₁₉NO₃: C, 63.98; H, 8.50; N, 6.22. Found: C, 63.71; H, 8.51; N, 6.15.

Alcohol (6)

Ketone 5 (1.35 g, 6.00 mmol) was dissolved in 2.5:1 THF/MeOH (70 ml) and cooled to 0° C. CeCl₃ (2.69 g, 7.20 mmol) was added, followed by NaBH₄ (0.46 g, 12 mmol). After stirring 1 h at 0° C., the reaction was quenched with NH₄Cl (15 mL), diluted with EtOAc (100 mL), washed with NH₄Cl (2×20 mL), brine (20 mL), dried on MgSO₄ and concentrated. Chromatography on silica with 20% EtOAc in hexane yielded 1.36 g (100%) of product as a white solid. ²H NMR δ 5.66(m, 1H), 4.89 (br,s, 1H),4.31 (d,J=5.5, 1H),3.38(m, 1H), 3.13 (m, 1H), 2.31 (m, 4H), 1.88 (m, 2H), 1.43 (s, 9H) ¹³C NMR δ 156.7, 144.6, 126.4, 79.6, 70.9, 45.3, 32.3, 31.9, 28.4, 23.4. Anal. Calcd for: C₁₂H₂₁NO₃: C, 63.41; H,9.31; N, 6.16. Found: C, 63.63; H, 9.47; N, 6.09.

Ester (7)

To a solution of alcohol 6 (12 mg, 0.053 mmol) and pyridine (13.3 μL, 0.165 mmol) in THF (0.1 mL) was added a solution of t-butyldimethylsilyloxyacetyl chloride (Bischofberger N, Waldmann H, Saito T, Simon E S, Lees W, Bednarski M D, Whitesides G M: Synthesis of Analogues of 1,3-Dihydroxyacetone Phosphate and Glyceraldehyde 3-Phosphate for Use in Studies of Fructose-1,6-diphosphate Aldolase. J. Org. Chem. 1988, 53:3457-3465) (12 mg, 0.055 mmol) in THF (0.1 mL) dropwise at 0° C. The reaction was stirred for 0.5 h at rt then diluted with 5 mL Et₂O, washed with 0.5 N HCl (2×0.4 mL), NaHCO₃ (1 mL), brine (1 mL), dried on MgSO₄ and concentrated. Chromatography with 5% EtOAc in hexanes on silica gave 14.5 g(67%) of ester7 as colorless oil. ¹H NMR δ 5.67 (s, 1H), 5.48 (br, s, 1H), 4.64 (br, s, 1H), 4.24 (s, 2H), 3.43 (m, 1H), 3.33 (m, 1H), 1.87 (m, 2H), 1.45 (s, 9H), 0.90 (s,9H), 0.08 (s, 6H). ¹³C NMR δ 171.2, 155.8, 139.9, 128.8, 79.6, 72.8, 61.8, 42.8, 32.4, 32.0, 28.4, 25.9, 25.6, 23.1, −5.4.

α-Hydroxy Acid (8)

To a solution of diisopropylamine (0.21 mL, 1.5 mmol) in THF (2.0 mL) was added n-butyl lithium (2.5 M in hexane, 0.54 mL, 1.3 mmol) at 0° C. The mixture was stirred for 15 min to generate LDA. Then a mixture of chlorotrimethyl silane (0.46 mL, 3.7 mmol) and pyridine (0.32 mL, 4.0 mmol) in THF (0.8 mL) was added dropwise to the LDA solution at −100° C. After 5 min, a solution of ester 5 (136 mg, 0.333 mmol) in THF (1 mL) was added dropwise and the reaction was stirred at −100° C. for 25 min then warmed slowly to rt over 1.5 h and heated to 45° C. for 1 h. The reaction was quenched with 1 N HCl (5.0 mL) and the aqueous layer was extracted with Et₂O (2×7 mL). The organic layer was dried on MgSO₄ and concentrated to give 106 mg (crude yield 78%) yellowish glassy oil. Without further purification, the product was dissolved in 0.8 mL THF. Tetrabutylammonium fluoride (261 mg, 1.00 mmol) in THF (0.5 mL) was added at 0° C., stirred at 0° C. for 5 min then at rt. for 1 h. The reaction was quenched with 0.5 N HCl (2 mL), extracted with EtOAc (5 mL), dried on MgSO₄ and concentrated. Chromatography with 5% methanol in CHCl₃ on silica gave 46.2 mg (52%) of α-hydroxy acid 8 as yellowish oil. ¹H NMR (DMSO-d₆) δ 6.81, (br, s, 1H)), 5.31 (br, s, 1H), 3.84 (d, J=5.8, 1H) 3.48 (m, 2H), 3.16 (t, J=8.5, 1H) 2.64 (m, 1H), 2.27 (m, 1H), 2.12 (m, 1H), 1.70 (m, 2H), 1.58-1.42 (m, 2H), 1.37 (s, 1H). ¹³C NMR (DMSO-d₆) δ 175.4, 156.0, 144.4, 120.2, 79.7, 78.0, 73.7, 58.1, 47.4, 29.8, 28.9, 24.5, 23.6, 24.5, 23.6, 19.8, 14.1.

Acid (9)

Lead tetraacetate (78 mg, 0.17 mmol) in CHCl₃ (0.4 mL) was added dropwise to a solution of acid 8 (45.6 mg, 0.16 mmol) in EtOAc (2.2 mL) at 0° C. The reaction was stirred for 10 min, then quenched with ethylene glycol (0.6 mL), diluted with EtOAc (20 mL), washed with H₂O (4×2 mL) and brine (2 mL), dried on Na₂SO₄, and concentrated to give 38 mg (100% crude yield) of aldehyde as yellow oil. The product was dissolved in acetone (4.8 mL) and cooled to 0° C. Jones reagent (2.7 M H₂SO₄, 2.7 M CrO₃; 0.12 mL, 0.32 mmol) was added dropwise. The reaction was stirred at 0° C. for 0.5 h, quenched with isopropyl alcohol (0.5 mL), and stirred for 10 min. The precipitate was filtered out, and the solvent was evaporated. The residue was extracted with EtOAc (3×5 mL), washed H₂O (1.5 mL) and brine (1.5 mL), dried on Na₂SO₄, and concentrated. Chromatography on silica with 40% EtOAc and 0.1 acetic acid in hexane gave 12.5 mg (31%) of acid 9 as a white solid. ¹H NMR (DMSO-D₆) δ 12.16 (br, s, 1H), 6.93 (t, J=5.4, 1H),5.37 (s, 1H), 3.50 (m,22H), 3.16 (t,J=7.3, 1H), 2.29 (m, 1H), 2.22 (m, 1H), 1.80 (m, 3H), 1.55(m, 1H), 1.37(s, 9H). ¹³C NMR(DMSO-D₆) δ 175.3, 156.1, 142.9, 120.8, 78.1, 49.5, 30.1, 29.2, 28.8, 25.0. Anal. Calcd for: C₁₃H₂₁NO₄: C, 61.16; H, 8.29; N, 5.49. Found: C, 61.11; H, 8.25; N, 5.48.

Amide (10): 1-Hydroxybenzotriazole (HOBt, 191.6 mg, 1.25 mmol), N-[(1H-benzotriazol-1-yl)(dimethylamino)methylene]-N-methylmethanaminiun hexafluorophosphate N-oxide (HBTU, 473.8 mg, 1.25 mmol), DIEA (3225/5 mg. 2/5 mmol) and acid 7 (119.6 mg, 0.5 mmol) were dissolved in DMF (25 mL), 4-Hydroxyproline methyl ester hydrochloride salt (224.5, 1.25 mmol) was added. The reaction mixture was stirred at rt for 1 h, then diluted with EtOAc (75 mL), washed with H₂O (3×25 mL), NaHCO₃ (25 mL), brine (25 mL), dried on MgSO₄ and concentrated. Chromatography with 50% EtOAc in hexanes yielded 110 mg of syrup.

Amine (11): Amide 10 (110 mg, 0.302 mmol) and triethylsilane (87.79 mg, 0.755 mmol) were dissolved in 25% TFA in DCM and stirred for 0.5 h at rt. Solvent was removed by evaporation. Remaining TFA and triethyl silane was removed by vacuum. Without further purification, the residue was dissolved in 2 mL DCM, tert-butyldimethylsilyl chloride (91 mg, 0.604 mmol) and imidazole (82 mg, 1.208 mmol) were added. The reaction mixture was stirred at room temperature for 4 h then diluted with EtOAc, washed with NaHCO₃ (2×7 mL), H2O (7 mL), dried on MgSO₄ and concentrated. Chromatography on silica gel with 15% MeOH in chloroform gave 81 mg (67.7%) colorless oil.

Acid (12)

To a solution of amine 11 (80 mg, 0.2 mmol) in THF (1.2 mL) was slowly added a solution of potassium hydroxide in 1:2 MeOH: H2O (0.6 mL) at −10° C. After stirring for 1 h at 0° C., the reaction was diluted with 5 mL THF, acidified with 1 N HCl (0.21 mL), dried over MgSO₄ and concentrated. Chromatography on silica gel with 15% MEOH in CHCl₃ yielded 50 mg (yield 65.3%) of colorless oil.

(Gly-Pro-Pro)ₙ polymer: The monomer H-Gly-Pro-Pro-OH (590 mg, 2.19 mmol) was dissolved in 3 mL NMP at 0° C. HBTU (1.67 g, 4.38 mmol) and HOBt (695 mg, 4.54 mmol) were added and the resulting solution was stirred at 55° C. for 3 days. After cooled to r.t, the solvent was evaporated by vacuum and yellowish oily liquid mixture was obtained. ¹H NMR (crude CDCl₃ with TFA): δ3.70, 3.53, 3.36, 3.28, 3.15, 2.92, 1.52-1.22; MALDI: highest MW found: 2669.4, GPC: polymer peak showed.

Polymer (Gly-Ψ[(E)CH═C]-Pro-Hyp)ₙ mimic (1a): The monomer H-Gly-Ψ[(E)CH═C]-Pro-Hyp-OTBS-OH (17 mg, 0.044 mmol) was dissolved in 0.5 mL DMF at 0° C. HATU (76.9 mg, 0.20 mmol) and DIEA (0.04 mL, 0.23 mmol) were dissolved in 1.0 mL DMF at 0° C. and the solution was added to the monomer solution. The resulting solution was stirred at 50° C. for 12 hrs and then it was cooled to r.t. The solvent was evaporated by vacuum and dark red oily liquid mixture was obtained. ¹H NMR (crude in CDCl₃): δ7.17, 3.62, 3.42, 3.31, 3.20, 3.16-2.84, 1.42-1.15.

Thus, a polymer (1a) has been synthesized by polymerization of H-Gly-Ψ[(E)CH═C]-Pro-Hyp(OTBS)-OH monomer 12. Other polymers likewise may be synthesized by the inventive polymerization methods, in other cases of monomers mentioned herein because in those cases, too, the alkene being situated in the middle of the molecule would not be expected to affect the reactivity.

While the invention has been described in terms of its preferred embodiments, those skilled in the art will recognize that the invention can be practiced with modification within the spirit and scope of the appended claims.

1. A polymeric material which comprises at least one peptidomimetic selected from the group consisting of:
(Gly-Ψ[(E)CH═C]-Xaa-Yaa)ₙ (1A)
(Gly-Xaa-Ψ[(E)CH═C]-Yaa)ₙ (1B)
(Gly-Xaa-Yaa-Ψ[(E)CH═CH])ₙ (1C)
(Gly-Ψ[(E)CH═C]-Xaa-Ψ[(E)CH═C]-Yaa)ₙ (2A)
(Gly-Xaa-Ψ[(E)CH═C]-Yaa-Ψ[(E)CH═CH])ₙ (2B)
(Gly-Ψ[(E)CH═C]-Xaa-Yaa-Ψ[(E)CH═CH])ₙ (2C)
and
(Gly-Ψ[(E)CH═C]-Xaa-Ψ[(E)CH═C]-Yaa-Ψ[(E)CH═CH])ₙ (3)
wherein Xaa and Yaa may be the same or different and represent a natural amino acid, Hyp or Flp;
n means an integer.

2. The polymeric material of claim 1, wherein n is 10 or more.

3. The polymeric material of claim 1, wherein the peptidomimetic comprises:
(Gly-Ψ[(E)CH═C]-Xaa-Yaa)ₙ (1A)
wherein Xaa is Pro and Yaa is Hyp.

4. The polymeric material of claim 1, comprising a block polymer as follows: 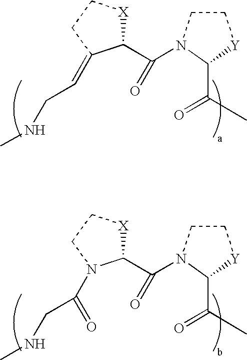
wherein a and b are integers between about 5 and 125, wherein a and b may be the same or different.

5. The polymeric material of claim 1, comprising a block copolymer of a peptidomimetic with a natural peptide.

6. The polymeric material of claim 1, comprising a monomer as follows:

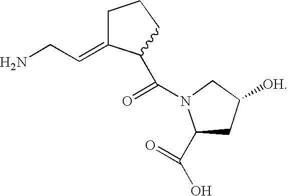

7. The polymeric material of claim 1, the polymeric material mimicking collagen.

8. The polymeric material of claim 7, wherein the polymeric material is biocompatible and upon insertion into a region in a living patient where collagen at a previous time had been disposed, the inserted polymeric material provides at least one property of natural collagen.

9. A product comprising a polymeric material which is not naturally occurring, comprises alkene bonding and has a triple helix rope-like structure.

10. The product of claim 9, wherein the polymeric material comprises at least one selected from the group consisting of: 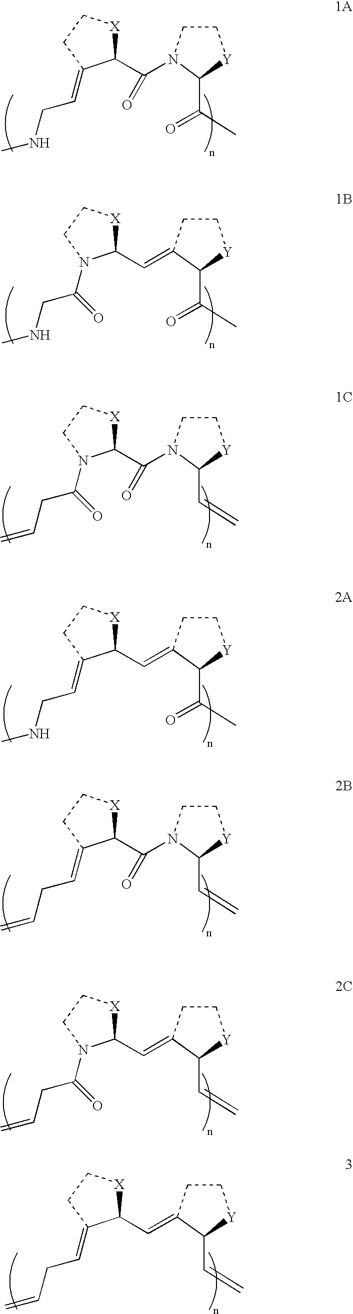
wherein n means an integer.

11. The product of claim 10, wherein n is 10 or more.

12. The product of claim 10, wherein the polymeric material has one or more selected from the group consisting of: greater stability than natural collagen, and greater collagenase-resistance than natural collagen; greater ability to fold than natural collagen.

13. The product of claim 10, implanted or injected into a living organism.

14. The product of claim 10, having biology purity suitable for use in a living human patient.

15. The product of claim 10, not capable of producing a problematic immunologic reaction when injected into living human patients.

16. A method of tissue replacement in a living organism, comprising: delivering into the living organism the product of claim 1.

17. A method of hip replacement, comprising: disposing in a living organism the product of claim 1.

18. A biocompatible adhesive formed by the product of claim 1.

19. A method of biomineralization, comprising delivering into a living organism the product of claim 1.

20. A method of drug delivery, comprising: disposing in a living organism the product of claim 1 wherein the product comprises a drug.

21. A method of synthesizing collagen-like peptides, comprising polymerization of a H-Gly-Ψ[(E)CH═C]-Pro-Hyp-OH monomer.

22. The synthesis method of claim 21, including polymerizing tripeptide units.

23. The synthesis method of claim 21, wherein a (Gly-Pro-Hyp)ₜ polymer is synthesized wherein t is a number of repeating units of about 10 to 160.

24. The synthesis method of claim 21, wherein a polymer comprising (Gly-Pro-Hyp) repeating units and having molecular weight of about 40,000 is synthesized.

25. The polymeric material of claim 1, wherein the peptidomimetic comprises:
(Gly-Ψ[(E)CH═C]-Xaa-Yaa)ₙ (1A)
wherein Xaa is Pro and Yaa is Pro.
